(19) United States
(12) Patent Application Publication
(10) Pub. No.: US 2017/0356901 A1
(43) Pub. Date: Dec. 14, 2017

(54) T CELL-BOUND CYTOKINE ASSAY FOR ANTIGEN-SPECIFIC TOLERANCE

(71) Applicant: Wisconsin Alumni Research Foundation, Madison, WI (US)

(72) Inventors: William James Burlingham, Madison, WI (US); Dario Alberto Vignali, Pittsburgh, PA (US)

(21) Appl. No.: 15/618,648

(22) Filed: Jun. 9, 2017

Related U.S. Application Data
(60) Provisional application No. 62/348,653, filed on Jun. 10, 2016.

(51) Int. Cl. G01N 33/50; G01N 33/74; G01N 33/68; G01N 33/564

(52) U.S. Cl. CPC G01N 33/5091; G01N 33/564; G01N 33/74; G01N 33/6869; G01N 2800/245; G01N 2333/495; G01N 2333/54

(57) ABSTRACT

In vitro methods for detecting and measuring an antigen-specific regulatory T cell response are described. In particular, there is provided, for example, a method of detecting a change in surface expression of particular T cell markers in T cells obtained from the subject as a way to detect induced immune suppression in response to exposure to a particular antigen or plurality of antigens.

### CROSS-REFERENCE TO RELATED APPLICATIONS

This application claims the benefit of U.S. Provisional Application No. 62/348,653, filed Jun. 10, 2016, which is hereby incorporated by reference in its entirety for all purposes.

### STATEMENT REGARDING FEDERALLY SPONSORED RESEARCH OR DEVELOPMENT

This invention was made with government support under AI066219 awarded by the National Institutes of Health. The government has certain rights in the invention.

### BACKGROUND

Establishment of tolerance to a well-functioning transplant with limited specific inductive therapy is a major goal of organ transplantation. Such tolerance manifests as drug-free allograft acceptance, meaning that a transplant recipient exhibits tolerance to alloantigens (antigens present in members of the same species and used by the immune system to distinguish self from non-self) derived from the transplanted cells, tissue, or organ. However, drug-free allograft acceptance is rarely encountered in transplant recipients. Research on those recipients who have become tolerant of their grafts has shown that these patients have an active suppression of their immune response to the donor alloantigens. In other words, tolerance to specific antigens derived from donor tissue was associated with the absence, or a reduction in intensity of, one or more immune responses to a specific antigen, particularly those responses responsible for the detrimental impact on the recipient. This phenomenon, known as the “bystander effect,” appears to be mediated by T-regulatory cells (“Tregs”) and involves antigen-specific suppression of an immune response in other cells exposed to the same antigen. While Tregs were initially defined by their role in maintaining tolerance to “self” antigens, it is clear that Tregs also play an important role in suppressing immune responses directed against alloantigens expressed on transplanted organs and tissues.

The potential to induce antigen-specific tolerance for transplant recipients and patients with autoimmune disorders has been explored as way to halt pathogenic autoimmune responses and to prevent graft rejection while avoiding potentially severe side effects associated with many immunotherapies. However, few assays exist to measure the regulatory portion of an immune response. In 2000, Burlingham and coworkers described a trans vivo ‘delayed-type’ hypersensitivity (tv-DTH) assay to detect linked suppression by injecting the antigen into the footpad of the animal and measuring swelling of the footpad at defined time points. In the intervening 15 years, others have tried with little success to develop in vitro non-animal assays to measure and model human DTH responses, but most of these non-animal assays measure antigen non-specific responses using whole donor cells as a source of antigens. Accordingly, there remains a need in the art for efficient, purely in vitro methods for detecting and quantifying antigen-specific regulatory immune responses linked to the immune suppression. In addition, there remains a need for biomarkers of tolerance in human disease.

### BRIEF SUMMARY

In the interest of providing a clear and concise summary, the following description references certain exemplary aspects and embodiments. Persons of ordinary skill in the art will, in view of the teachings in this application, readily recognize and appreciate that other aspects, embodiments, configurations, and variations of the technology disclosed herein are possible and that the exemplary aspects and embodiments described in this summary or elsewhere in this application are neither limiting nor exhaustive.

In a first aspect, provided herein is an in vitro method of detecting antigen-specific immune suppression in a subject, the method comprising (a) culturing T cells of the subject for about 24 hours in the presence of one or more target antigens; and (b) detecting in the cultured T cells expression of a marker that that indicates antigen-specific regulatory T cell response in the subject, wherein detecting expression of the marker in the population of cultured T cells indicates antigen-specific immune suppression in the subject. The T cells can be obtained from a biological sample selected from the group consisting of lymph nodes, peripheral blood, and splenocytes. The marker can be selected from the group consisting of Ebi3 and TGFβ/LAP. In some cases, step (b) is carried out by measuring the proportion of cells positive for surface expression of Ebi3 among CD4-positive or CD8-positive T cells of the cultured T cells. In some cases, step (b) is carried out by measuring the proportion of cells positive for intracellular expression of TGFβ/LAP among CD4-positive or CD8-positive T cells of the cultured T cells. The one or more target antigens can comprise self-antigens and the antigen-specific linked immune suppression is associated with an autoimmune disease. The one or more target antigens can comprise alloantigens and the antigen-specific linked immune suppression is associated with a cell, tissue, or organ transplant.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents and alternatives. Reference should therefore be made to the claims recited herein for interpreting the scope of the invention.

### BRIEF DESCRIPTION OF THE DRAWINGS

The present invention will be better understood and features, aspects, and advantages other than those set forth above will become apparent when consideration is given to the following detailed description thereof. Such detailed description makes reference to the following drawings, wherein:

FIG. 1 is microscopic examination of surface Ebi3 expression by imaging flow cytometry after 24 hour culture of lymph node cells from 35 day old CBA-tolerized B6 Yellow Fluorescent Protein (YFP)-Foxp3/TdTomatoRed-Ebi3 dual reporter mice with a crude CBA antigen (CBA-Ag) prep. (left) A Foxp3⁺ T cell that is also surface Ebi3⁺. While the Ebi3 protein staining was evenly distributed over the cell surface, Td-TomatoRed evidences Ebi3 gene transcription. (right) The most common surface phenotype is shown in a transcriptionally-negative Ebi3^{neg} cell. A punctuate pattern of surface Ebi3 was observed, which suggests that the cells have acquired Ebi3⁺ exosomes from an exosome-producing cell.

FIG. 2 demonstrates surface expression of TGFβ/LAP (latency-associated peptide) (“sTGFβ/LAP”), after overnight culture with specific tolerogen. (left) A rare sTGFβ/LAP⁺, CD4+ T cell that is also Foxp3⁺. The TGFβ/LAP protein staining is unevenly distributed over the entire cell surface in the absence of an Ebi3+ nuclear signal. (right) The most common surface phenotype is shown in a Foxp3^{neg}Ebi3^{neg} cell with polar, intense surface TGFβ/LAP staining.

FIG. 3 demonstrates (top histograms) a flow cytometry gating strategy to gate, from left to right, lymphocytes, single cells, T cells, and CD4⁺ T cells; and (bottom histograms) flow cytometry data for CD4⁺ T cells expressing surface Ebi3 (“sEbi3”) as determined by V1.4F5.29 antibody staining or surface TGFβ/LAP (“sTGFβ/LAP”) relative to intracellular Ebi3 mRNA synthesis. Note that sEbi3 expression is about eight (8) times greater than sTGFβ/LAP expression. FSC-A: forward scatter area; SSC-A: side scatter area: FSC-H: forward scatter pulse height; CD3: CD3+ T cells; CD4: CD4+ T cells.

FIG. 4 demonstrates percentages of CD4+ T cells as determined by (from left to right) intracellular Ebi3 (iEbi3) gene transcription by Td-TomatoRed signal; surface-bound Ebi3 (“sEbi3”) protein expression by V1.4F5.29 staining; and surface-bound TGFβ/LAP staining (“sTGFβ/LAP”). Lymph node cells were harvested from CBA-tolerized or DBA-tolerized B6/YFP-Foxp3/Td-TomatoRed-Ebi3 dual marker mice, tested 35 days after donor-specific transfusion (DST)+anti-CD154 mAb tolerization. Each data point represents a single mouse. *p<0.05 ***p<0.0001. Media: media control.

FIG. 5 compares the percentage of linked suppression as determined using tv-DTH or by detecting expression of intracellular Ebi3, sEbi3, and sTGFβ/LAP. The x-axis of each graph plots the percentage of marker-positive cells over percentage of media control, and the y-axis of each graph plots the percentage of inhibition by tv-DTH.

FIG. 6 demonstrates the results of a T-CBC assay detecting tolerance induced by “self-antigen” human collagen type 5 (Col V) as detected by surface expression of sTGFβ/LAP. Cells used in the assay were normal peripheral blood mononuclear cells (PBMCs).

FIG. 7 demonstrates that human sEbi3 T-CBC test results correlate with linked suppression values obtained by the trans-vivo DTH (mouse footpad swelling) inhibition assay in a patient with metastable tolerance. The graph compares (i) surface Ebi3⁺ (sEbi3⁺) cells as a percentage of the total number of CD4 T cells at 3 different time-points and (ii) and as a percentage of linked suppression (LS) at each time-point for a kidney transplant patient exhibiting metastable tolerance.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION

All publications, including but not limited to patents and patent applications, cited in this specification are herein incorporated by reference as though set forth in their entirety in the present application.

The methods and compositions provided herein are based at least in part on the Inventors' development of an assay referred to herein as the T cell assay for cell-bound cytokines (T-CBC). The T-CBC assay is an in vitro assay capable of (or configured to) detecting and measuring antigen-specific regulatory immune responses linked to the immune suppression, where the response is specific for self-antigens (in the case of autoimmune disease or transplant-induced autoimmunity) or for donor alloantigens (in the case of a human leukocyte antigens (HLA) and/or transplant recipient). Without being bound to any particular theory or mechanism of action, the T-CBC assay is based on the principle that two distinct populations of antigen-specific Tregs produce cell-bound cytokines in response to stimulation by a particular antigen or tolerogen: (1) a population comprising Foxp3+ CD4 and CTLA4+ CD8 T cells; and (2) a population comprising Foxp3^{neg} CD4+ or CD8+ T cells that co-express CD39 or CD25^{low}. Cells of the first population (Foxp3+/CD4+ and CTLA4+CD8+ T cells) exhibit uniform surface expression of Ebi3 after encountering a specific antigen and antigen presenting cell (APC). Surface Ebi3+(sEbi3+) cells represent no more than 0.1-0.5% of total of the first population. Cells of the second population (Foxp3^{neg} CD4+ T cells or CD8+ T cells that co-express CD39 or (dimly) CD25) express the latent form of TGF-β/LAP, complexed with either latency-associated peptide (LAP) or latent TGF-β binding protein (LTBP), over a large portion of the cell surface, but the expression is not uniform. Like the sEbi3+ cell population, the population of Foxp3^{neg} CD4+ T cells or CD8+/CD39+ or CD8+/CD25^{low} T cells represents no more than 0.1-0.5% of total CD4+ or CD8+ T cells in a heterogeneous population of lymphocytes.

The in vitro methods described herein improve upon the trans-vivo linked suppression assay (also known as the trans vivo-DTH assay) by detecting the induced expression of particular cytokines, specifically IL-35 or its Ebi3 subunit or LAP-TGFβ complex following exposure of T cells to a specific target antigen or unknown antigens found, for example, in a cell lysate. Induced expression of these cytokines, both on the surface of the antigen-specific regulatory T cells, and in the case of IL-35, as acquired exosomes landing on the surface of a vastly greater number of “bystander” memory T cells, which are then subjected to suppression and acquisition of an ‘exhaustion’ phenotype, replicates in vitro the suppression of footpad swelling observed when human cells are injected into a mouse footpad as part of the trans-vivo DTH assay. As used herein, the term “bystander” should be understood to refer to cells that do not produce exosomes but rather passively acquire exosomes from exosome producers. An increase in the T-CBC readout (e.g., evidence of induced expression of cell-bound cytokines) is thus seen as a proxy for linked suppression, while the absence of any change in expression (e.g., increase or decrease) of a T-CBC value indicates that the T cell donor (e.g., human subject) does not exhibit antigen-specific immune suppression. Advantageously, the T-CBC assay can be performed on the same 24-hour time scale as the trans-vivo DTH (“tv-DTH”) assay, but requires far fewer cells from the tested individual and avoids the need for vertebrate animals. Even more advantageously, the methods provided herein are amenable to automation and miniaturization for a variety of applications including clinical laboratory uses.

Accordingly, in a first aspect, provided herein is an in vitro method for detecting and/or quantifying antigen-specific linked suppression attributable to a regulatory T cell (“Treg”) response, where the method includes incubating T cells or a T-cell containing mixture obtained from an individual with a target antigen or plurality of antigens and detecting evidence of immune suppression specific for that antigen in the incubated T cells or T cell mixture. In an exemplary embodiment, the method comprises or consists essentially of (a) culturing T cells of the subject for about 24 hours in the presence of one or more target antigens; and (b) detecting among CD4-positive or CD8-positive T cells of the cultured T cells surface expression of a marker that that indicates antigen-specific regulatory T cell response in the subject, wherein detecting expression of the marker in a population of the cultured T cells indicates antigen-specific immune suppression in the subject. In some cases, the marker is TGFβ, IL-35, or Ebi3, which is secreted in exosomes from Tregs and bound to the surface of a population of bystander CD4+/Foxp3-negative (Foxp3^{neg}) non-Tregs (meaning, CD4+ T cells that are not Tregs).

In a particular embodiment, the method comprises the following steps: (a) a single cell suspension of mononuclear cells obtained from the spleen, lymph nodes, or peripheral blood of a subject (e.g., human patient) is cultured for a predetermined length of time in the presence of a specific antigen (e.g., a particular self-antigen) or unknown antigens in a donor cell lysate; (b) harvesting and optionally washing the cultured cells to remove unbound or loosely bound proteins; (c) sorting harvested cells (e.g., flow cytometry with CD3/4 or CD3/8 gating); and (d) detecting expression of a marker that indicates a suppressive (regulatory) response that is specific to the particular antigen or antigen(s). In some cases, the predetermined length of time is an hour or more (e.g., about 1, 2, 4, 6, 8, 10, 12, 24, 28, 32, 36 hours).

In certain embodiments, expression of a marker that indicates antigen-specific immune suppression is detected using an antibody having specificity for TGFβ, IL-35, or the Ebi3 (β subunit) and IL-12 (α subunit) components of IL-35. For example, TGF-β can be detected using an antibody having specificity for a form of TGF-β. TGF-β is secreted by many cell types in a latent form in which it is complexed with two other polypeptides, latency-associated peptide (LAP) and latent TGF-beta binding protein (LTBP). Accordingly, in some cases, expression of TGF-β comprises detecting surface expression of TGF-β complexed with polypeptide LAP (“TGFβ/LAP”). In some cases, the marker is IL-35 (or components thereof) and/or TGFβ-LAP bound to the surface of sorted T cells, and detecting can comprise incubating sorted cells in the presence of an anti-TGFβ-LAP antibody, an anti-IL-35 antibody, or an anti-Ebi3 antibody.

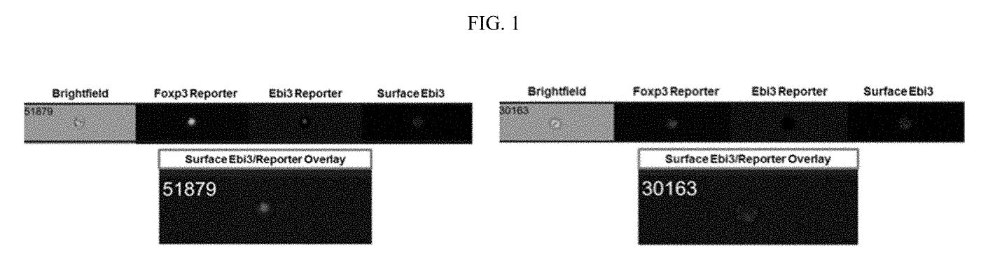

In some cases, the method comprises using a unique Ebi3 monoclonal antibody (V1.4F5.29) which does not neutralize IL-35 or IL-27, as described by Collison et al. (Nature Immunol. 11(12):1093 (2010)). Collison describes clones V1.4F5.29, V1.4H6.25 and V1.4C4.22, obtained by immunizing Ebi3^{-/-} mice using recombinant mouse Ebi3, and chosen for their utility in immunoprecipitation, immunoblot analysis, and/or specific neutralization of IL-35 bioactivity. Preferably, an anti-Ebi3 antibody such as clone V1.4H6.29 (Collison et al., 2010) is used to detect expression of Ebi3 in exosomes released by T cells following incubation with target antigens. As used herein, the term “exosome” refers to membranous, extracellular nano-sized vesicles (“nanovesicles”) released by a specialized endocytic compartment of antigen-presenting cells. Exosomes range in size from about 30 nm to 100 nm and contain RNA, DNA, and proteins from their cell of origin. T cells having cell surface expression of Ebi3 are a different subset of cells from the CD4+/CD25+ Tregs that produce the exosomes. T cells exhibiting surface expression of Ebi3 are merely bystander cells that passively acquire the exosomes released by exosome-producing CD4+/CD25+ Tregs. Treg producers of Ebi3⁺ exosomes may be interrogated by their distinctive Ebi3 staining pattern (i.e., uniform, not punctate, staining) and, in some cases, nuclear expression of forkhead box P3 (Foxp3), which is a useful biological marker of Tregs (Sakaguchi et al., Nat Rev Immunol. 2010; 10(7):490-500). Referring now to FIG. 1, Ebi3 can be detected on the surface of Ebi3-transcriptionally active cells (left), as well as on Ebi3-transcriptionally inactive bystander cells (right). FIG. 1 demonstrates that in Ebi3-transcriptionally active Foxp3⁺ T cells, Ebi3 protein expression is uniform over the entire cell surface. The surface membranes of cells that do not themselves express Ebi3 (“non-Ebi3-producer cells”) exhibit punctate expression of round Ebi3⁺ exosome-like structures.

As used herein, the term “T cell” refers to cells of the immune system that function as a biodefense system against various pathogens and includes CD4-positive (CD4⁺) helper T cells and CD8-positive (CD8⁺) cytotoxic T cells, where the former relates to promoting immune response and the latter relates to excluding virus-infected cells and tumor cells. As used herein, the term “T cell-containing cell mixture” refers to a cell population or cell composition that comprises T cells. A T cell-containing cell mixture preferably comprises one or more specific T cell types (e.g., CD4+ helper T cells, CD8+ cytotoxic T cells). CD4+ helper T cells are further classified into regulatory T (“Treg”) cells and conventional T helper (Th) cells. As used herein the term “Treg” refers to regulatory T cells, either singular or plural. While Th cells control adaptive immunity against pathogens and cancer by activating other effector cells such as CD8⁺ cytotoxic T cells, B cells, and macrophages, Tregs function to suppress potentially deleterious activities of Th cells.

In some cases, the target antigens incubated with T cells or a T cell-containing cell mixture are specific, known antigens. A suitable target antigen can be any substance, molecule, microorganism, or fragment thereof that can bind to cells and/or molecules of the immune system and elicit an immune response. Target antigens include proteins, peptides, carbohydrates, nucleic acids, fragments of any of the foregoing, and chemicals. Target antigens can be isolated from natural sources or produced and/or modified in the laboratory, and include self-antigens, alloantigens, and tolerogens. Alloantigens include human leukocyte antigens (HLA) of a donor or prospective donor in a transplant setting. Self-antigens may include autoantigens of a target tissue or solid organ. As used herein, the term “tolerogen” refers to any substance (e.g., an antigen) used to induce tolerance, as distinguished from an immunogen, that induces immunity. By the terms “tolerogenic” or “tolerogenic activity” it is meant that a response of immunological tolerance is induced by an antigen or antigenic substance or an activity that results in the induction of immunological tolerance toward an antigen or antigenic substance. The term “tolerance” as used herein refers to a decreased level of an immune response, a delay in the onset or progression of an immune response and/or a reduced risk of the onset or progression of an immune response. Donor antigens can be prepared from peripheral blood mononuclear cells (PBMCs) or splenocytes and can be used to control for non-T-CBC-mediated responses, as explained below. In some cases, antigens are purified human leukocyte antigens (HLA). For self-antigen-specific Treg cell detection, the antigen preparations may be purified proteins from commercial sources.

In some cases, target antigens may be contacted to T cells in the form of a crude antigen-containing lysate. For example, a crude cell lysate can be used if the goal is to detect alloantigen-specific tolerance. In some cases, crude cell lysates provide donor antigens, but the specific alloantigen(s) that drive donor-specific Treg cell responses in the recipient are unidentified. Crude cell lysates comprise nanovesicles and soluble protein, allowing for both indirect and semi-direct T cell engagement. For example, a crude cell lysate for detecting alloantigen-specific tolerance in accord with the methods provided herein can be prepared by centrifuging a sonicate of 10×10⁶ donor cells such as peripheral blood mononuclear cells (PBMCs) or splenocytes at 10,000×g in the presence of at least one protease blocker and then collecting. In some cases, the resulting supernatants were used at a concentration of about 4×10⁷ cell equivalents per mL. In terms of purified HLA protein, the crude cell lysate can comprise about 1 μg to about 50 μg of HLA antigen.

In certain embodiments, the method includes incubating T cells or a T cell-containing cell mixture in the presence of a target antigen or plurality of antigens for a predetermined interval. A period of incubation may vary widely. In some cases, the cells and target antigen(s) are incubated for about 24 hours. In other cases, incubation may be for an interval of from a few minutes (e.g., 5 minutes, 10 minutes) to an interval of a few hours (for example, 2 hours, 3 hours, 4 hours) to 8 or more hours (e.g., 8 hours, 10 hours, 12, hours, 18 hours, 24 hours, or more). Preferably, T cells (e.g., from a T cell-containing cell mixture) are cultured in the presence of a target antigen or antigens in a culture medium. In some cases, the cell culture medium is DMEM. In some cases, the culture medium is supplemented with fetal bovine serum. In some cases, the culture medium is supplemented using about 1% to about 20% fetal bovine serum.

T cells appropriate for use according to the methods provided herein include mononuclear cells obtained from human blood and include, without limitation, peripheral blood mononuclear cells (PBMCs, also known as peripheral blood mononuclear lymphocytes (PBLs), bone marrow-derived mononuclear cells, and cord blood mononuclear cells (CBMCs). In some cases, T cells are obtained from biological samples comprising lymph node tissue, bone marrow, and/or splenocytes. Any appropriate method of isolating T cells from biological samples of the subject can be employed. A T cell-containing cell mixture or a purified T cell population can be obtained, for example, from splenocytes, lymph nodes, or peripheral blood mononuclear cells (PBMCs) of the subject or by leukapheresis of peripheral blood from a human subject. As used herein, the term “purified T cell population” refers to T cells isolated, separated, or otherwise removed from the blood or a leukocyte milieu (e.g., obtained by leukapheresis), whereby isolated or separated T cells exist in a physical milieu distinct from that in which they occur in vivo. The term does not imply any particular degree of purity, and the absolute level of purity is not critical. Those skilled in the art can readily determine appropriate levels of purity for use according to the methods provided herein. Those skilled in the art are familiar with many established protocols for isolating PBMCs. Human peripheral blood may be drawn conveniently via venipuncture. Isolation of PBMCs can be aided by density-gradient separation protocols, usually employing a density-gradient centrifugation technique such as Ficoll®-Hypaque or Histopaque® for separating lymphocytes from other formed elements in the blood. Preferably, PBMC isolation is performed under sterile conditions. Alternatively, cell elutriation methods may be employed to separate mononuclear cell populations. Advantages of the cell elutriation method include sterility and efficiency.

By way of example, an exemplary protocol for obtaining T cells suitable for the methods provided herein comprises the following steps: (a) collect blood into ACD (Acid Citrate Dextrose) tubes; (b) isolate PBMC from fresh human peripheral blood using Lymphocyte Separation Medium according to standard methods; (c) wash the PBMC three times with PBS to remove contaminating platelets. Platelets were found to interfere with trans-vivo DTH assay. Maximal allowable platelet contamination of PBMC preparation is ≦1×10⁷/injection; and (d) if there is a noticeable red blood cell contamination, perform lysis of red cell using ACK lysis buffer after first wash. Remove ACK buffer by washing 2 times with PBS.

By way of example, an exemplary protocol for obtaining a T cell lysate for a plurality of alloantigens comprises the following steps: (a) isolate PBMC from donor peripheral blood using the procedure described above; (b) resuspend donor PBMC in PBS at a concentration of 120×10⁶ cells/ml (4×10⁶ cells/30 μl); (c) add 1 μM PMSF to the mixture to prevent protein degradation; (d) sonicate the cell suspension using seven 1-second pulses with a 2 mm-probe sonicator. (Note: Keep the material cold and avoid excessive bubbles. If foaming occurs, let the cell suspension sit on ice for a 2-3 min.); (e) verify the disruption of >90% of the cells using a hemocytometer; (f) centrifuge the mixture at 14,000 rpm at 4° C. for 20 min in refrigerated microfuge; and (g) transfer supernatant to a new 2.0 ml safe-lock tube and determine protein concentration.

In some cases, expression of a marker that indicates an antigen-specific Treg response following co-incubation of T cells as described herein is compared to expression of the markers in T cells ex vivo. Biological markers and methods useful for qualitatively and/or quantitatively evaluating the incubated cells of step (a) for evidence of an antigen-specific immune response are described herein and in the Examples that follow. If an antigen-specific Treg response is detected based on such qualitative and/or quantitative analyses, the result indicates that the T cell donor (i.e., the test subject) exhibits a robust regulatory response to the specific tolerogen or target antigen. In such cases, it may be advisable to consider lowering or discontinuing administration of an immune-suppressive drug therapy to the subject. Molecular markers of Tregs include CD25, cytotoxic T lymphocyte-associated antigen 4 (CTLA-4), forkhead/winged-helix transcription factor box P3 (Foxp3), glucocorticoid-induced tumor necrosis factor receptor family-related gene (GITR), CD127, and lymphocyte activation gene-3 (LAG-3).

Any appropriate method can be used to analyze incubated T cells for expression of a marker that indicates an antigen-specific Treg response. For example, analyzing can comprise identifying or sorting of regulatory T cell populations by flow cytometry, which may or may not involve fluorescence activated cell sorting (FACS). In some cases, analyzing comprises immunofluorescence staining or population microscopy. Antibodies to various cytokines (e.g., anti-Il-12, -EBi3, -Foxp3, -CD127, -CD4, -CD25, -CTLA-4 antibodies) can be used to sort by flow cytometry. Populations of cells can also be purified, e.g., using magnetic beads as described, e.g., in Bieva, et al. (1989) Exp. Hematol. 17:914-920; Hernebtub, et al. (1990) Bioconj. Chem. 1:411-418; Vaccaro (1990) Am. Biotechnol. Lab. 3:30. In preferred embodiments, imaging flow cytometry (“IFC”) is used to detect expression of a marker that indicates an antigen-specific Treg response and antigen-specific linked immune suppression in the individual among CD4-positive or CD8-positive T cells of the cultured T cells. In general, IFC combines flow cytometry with cell imaging to identify cellular events and evaluate protein expression at the level of a single cell in heterogeneous cell population, even in cases in which the level of expression of a particular protein of interest is low. IFC allows for the acquisition, identification, and statistical analysis of single cellular events but also cell aggregate content and cell-cell interactions based on fluorescent and morphological parameters. In some cases, it will be advantageous to fix and permeabilize cells to detect surface or intracellular expression of marker that indicates an antigen-specific Treg response.

By analyzing the cultured T cells for expression of a marker that indicates antigen-specific linked suppression of a regulatory T cell response in the subject, it can be determined what percentage of cells of the total population of cultured T cells express the marker. As used herein, the term “linked suppression” refers to a form of bystander suppression in which tolerated antigens and third-party antigens are presented by the same antigen-presenting cell (APC) or are co-expressed on the grafted tissue. Linked suppression is believed to be caused by the action of CD4+ Tregs and, more particularly, by the secretion of cytokines such as IL-35, IL-10, and TGFβ from alloantigen-specific Tregs. Suppression is said to be linked because, when the two antigens are co-localized, activity of a donor-specific Treg cell is able to inhibit the third party T effector cell.

As used herein, the terms “subject,” “patient,” and “individual” are used interchangeably and can encompass any vertebrate including, without limitation, humans, mammals, reptiles, amphibians, and fish. However, advantageously, the subject, patient, or individual is a mammal such as a human, or a mammal such as a domesticated mammal, e.g., dog, cat, horse, and the like, or livestock, e.g., cow, sheep, pig, and the like. In exemplary embodiments, the subject is a human. As used herein, the phrase “in need thereof” indicates the state of the subject, wherein therapeutic or preventative measures are desirable. Such a state can include, but is not limited to, subjects having a disease or condition such as cancer.

In certain embodiments, T cells obtained from a subject are cultured in the presence of a self-antigen, which may include human collagen type V or autoantigens of a target tissue or solid organ. Since collagen type V is subject to a natural Treg response, an increase in induced surface expression of Ebi3 following exposure of T cells of the subject to the self-antigen indicates antigen-specific (e.g., self-antigen specific) immune suppression. This increase can be compared to expression on T cells ex vivo. Where the self-antigen is contained in an autologous PBMC lysate and is not typically a target for immune regulation, the T-CBC assay should not reveal induced expression of IL-35 (Ebi3 subunit) or LAP-TGFbeta complex on the cell surface.

In some cases, T cells obtained from a subject are cultured in the presence of a donor alloantigen. If the patient is tolerant to the donor alloantigen, the methods described herein would reveal an increase in baseline level of surface Ebi3 expression on CD4 T cells ex vivo, and a further antigen-specific increase in T-CBC readouts after overnight culture.

In some cases, it may be advantageous to adapt the methods described herein for high-throughput, reproducible, and rapid detection, for example in a clinical setting. For example, Exclusion-Based Sample Preparation (ESP) can be used to automate and/or rapidly isolate analytes of interest from smaller sample sizes. Previously, it has been demonstrated that microfluidic culture systems can be used to accelerate paracrine signaling events and/or accelerate assay kinetics, due to the reduced culture volume and lack of dilutive convention currents. Thus, integrating microfluidic cell culture accelerates T cell response time, relative to the existing assay, without sacrificing sensitivity. Moreover, automated image analysis will enable one to distinguish patterns of uniform and patchy/exosome surface staining. With such adaptations, the complete T-CBC assay may be performed as a partially or completed automated system that is faster (e.g., wash steps can be performed without liquid transfer and accompanying cell loss) and uses an order of magnitude fewer host cells (e.g., 10⁵ cells rather than 10⁶ cells per assay) than the unadapted methods described herein.

In another exemplary embodiment, the assay can be configured to distinguish between absence of an effector response and presence of an immune regulatory response by including in the co-culture of antigen and T cells an agent that suppresses Treg function. Such agents include, but are not limited to, agents that suppress function of CTLA-4, IL-35, TGF-beta, extracellular ATPase CD39, or IL-10, such as blocking antibodies or receptor antagonists. As used herein, “suppress” means to lessen, diminish, or completely abrogate cell function. If the absence of expression of a marker that indicates antigen-specific Treg cell response is unaffected by the presence of such agent, such a negative response can be attributed to an absence of an effector response to the antigen of interest. If a negative expression response to the antigen (i.e., absence of expression of a marker that indicates antigen-specific Treg cell response) occurs in the presence (but not the absence) of the agent, however, the negative expression response can be attributed to an antigen-specific immune regulatory response suppressing the effector response.

Applications of the Methods

The methods provided herein are useful for detecting regulatory T cells, which cause bystander suppression. Bystander suppression of an antigen-specific regulatory response in the presence of donor antigen is characteristic of transplant recipients with accepted allografts. For example, CD4⁺CD25⁺ regulatory T cells (CD4⁺CD25⁺ Tregs), which constitute a small population of CD4⁺ T cells, are effective in suppressing the progression of antitumor immune responses, allograft rejection, and various autoimmune diseases. CD4⁺CD25⁺ Tregs also can be allopeptide/alloantigen-specific T regulatory cells and activated via their T-cell receptor (TCR) to become suppressive and to inhibit the proliferation of CD4+ or CD8+ T cells. Such alloantigen-specific Tregs are negative regulators of immune responses to alloantigen and, thus, critical for maintaining alloantigen-specific tolerance. Accordingly, the methods provided herein are useful to monitor transplant recipients for alloreactivity and to identify a subset of patients who could benefit from reduction of immunosuppression without elevated risk of rejection or deteriorating function of the transplanted organ. Other applications of the methods provided herein include, without limitation, application of the T-CBC assay for monitoring autoimmunity in a subject and for detecting response of the subject, such as a cancer patient, to immune therapy. For example, a decrease in induced expression of IL-35 (Ebi3 subunit) or LAP-TGFbeta complex on the T cell surface indicates that the subject responded to an anti-cancer immune therapy.

In another aspect, the methods provided herein are useful for detecting target antigen-specific immune regulatory responses as a method of screening or selecting individuals as suitable for vaccination with a particular antigen. In this manner, the invention disclosed herein also relates to methods for improved immunization strategies, i.e., administering a vaccine with one or more agents that suppress immune regulatory cell function, such as blocking antibodies or receptor antagonists, to enhance immune response to vaccination. The T cell-bound cytokine (T-CBC) assay can aid selection and monitoring of individuals likely to immunologically respond to such approach. The invention specifically contemplates identifying individuals in need of improved vaccination strategies. Improved vaccination strategies include administering a vaccine and an agent that suppresses immune regulatory cell function to improve immunity conferred by the vaccination. The vaccine and the agent can be administered to the subject at the same time or sequentially. Examples of agents that suppress immune regulatory cell function include agents that block the function of CTLA-4, IL-35, TGF-beta, extracellular ATPase CD39, or IL-10, such as blocking antibodies or receptor antagonists. In some cases, an individual may not exhibit a suppressor response immediately following vaccination but the suppressor response may instead develop over time. In such cases, it may be advantageous to administer a specific blockade treatment agent such as an anti-CTLA-4 or anti-IL-35 antibody.

Vaccines can elicit antigen-specific effector T (Teff) cells for anti-cancer treatment. In the case of DNA vaccines, an antigen is delivered to an individual in the form of DNA encoding the antigen, which is subsequently expressed, processed, and presented by antigen-presenting cells through MEW class I and can lead to potent CD8+ cytolytic T cells (Iwasaki et al., J. Immunol. 1997; 159(1):11-14; Chen et al., J. Immunol. 1998; 160(5):2425-32; Thomson et al., J. Immunol. 1998; 160(4):1717-23; Cho et al., Nat, Biotechnol. 2000; 18(5):509-14). DNA vaccines contain at least one gene encoding at least one peptide, protein, or protein fragment. Such gene can be part of a vector, such as a plasmid. The DNA can be packaged in vessels, such as liposomes or administered as “naked DNA.” DNA vaccines can be administered to an individual by any suitable method. For example, DNA vaccines can be administered via injection, such as intradermal or intramuscular injection. DNA vaccines can also be administered by using gene gun delivery, i.e., ballistically accelerating DNA that has been absorbed to suitable carrier micro-particles. Further administration routes include oral and topical application, such as by exposure to mucosal tissue.

Articles of Manufacture

In another aspect, the present invention provides articles of manufacture useful for identifying antigen-specific regulatory T cell responses. In preferred embodiments, the article of manufacture is a kit comprising one or more test antigens and one or more antibodies for detecting a test antigen-specific response when the one or more test antigens are incubated in the presence of T cells or a T cell-containing cell mixture obtained from the subject. In some cases, antigens may be provided in a kit as antigen preparations comprising purified proteins obtained, for example, from a commercial source. In some cases, the kit includes one or more markers for detecting an antigen-specific Treg cell response such as a monoclonal antibody having specificity for IL-35 or the components thereof (Ebi3 and IL-12), or an antibody having specificity for TGF-β complexed with polypeptide LAP (“TGFβ/LAP”).

Optionally, a kit can further include instructions for obtaining a T cell containing biological sample from a human subject and for performing a method as described herein to detect an antigen-specific Treg cell response using the subject's T cells.

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, preferred methods and materials are described herein.

In describing the embodiments and claiming the invention, the following terminology will be used in accordance with the definitions set out below.

As used herein, “about” means within 5% of a stated concentration range or within 5% of a stated time frame.

Having now described the invention, the same will be illustrated with reference to certain examples, which are included herein for illustration purposes only, and which are not intended to be limiting of the invention.

### EXAMPLES

### Example 1: Alloantigen-Specific Response Following Donor-Specific Transfusion (DST) and Co-Stimulatory Blockade Treatment in CBA-Tolerized Mice

To initially validate the T-CBC assay, we induced tolerance in C57BL/6 (“B6”) mice to CBA antigens using a donor-specific transfusion (DST) plus anti-CD154 (CD154 is also known as CD40 Ligand) tolerization method. The term “CBA antigens” refers to a lysate of spleen cells obtained from a mouse of the CBA strain and prepared by sonication. Following sonication and centrifugation at 10,000×g, the liquid supernatant is used as the source of CBA antigens. CD154 (aka CD40 Ligand) is a membrane glycoprotein and differentiation antigen transiently expressed on the surface of activated T cells. Through the binding of CD154 to CD40 on antigen presenting cells (APC) including B cells, monocytes/macrophages, and dendritic cells, it serves a crucial function in T cell-APC cognate interaction. CD154-interaction with CD40 transduces signals for T-dependent B cell activation and induces B cells to enter the cell cycle.

For review of the tolerization methods, see Tomita et al., Transplantation Direct 2016; 2:e73. Briefly, CBA (H-2^{k}) spleen cells were injected intravenously (i.v.) on day 0 into two types of dual reporter transgenic YFP-Foxp3/TdTomatoRed-Ebi3 B6 mice having an H-2^{b} background: (1) mice that expressed YFP under control of the Foxp3 promoter and expressed TdTomatoRed under control of the Ebi3 promoter, and (2) mice in which both reporters were present, but in which the Ebi3 gene was knocked out, such that Foxp3⁺ T cells [Ebi3^{loxp}×Foxp3^{Cre} F1] of these mice expressed no Ebi3 protein. To induce tolerance, a 125 mg dose of anti-CD154 (CD40 Ligand) monoclonal antibody (also known as MR1) was injected intraperitoneally (i.p.) into the mice on days 0, 2, and 4. Mice were sacrificed at days 14 and 35, and lymphoid tissues (spleen and lymph nodes) were harvested from the tolerized mice. After overnight (24 hour) culture of the lymphoid tissues with the specific tolerogen, we used flow cytometry and biological markers to detect IL-35 β subunit (Ebi3) gene transcription and surface protein expression.

On day 35, it was observed that the tolerized dual reporter mice (C57BL/6; H-2^{b} background) exhibited increased intracellular Ebi3 (“iEbi3”) signal in Treg cells and evidenced abundant sEbi3⁺ Foxp3^{neg} (non-Treg cells) cells (p<0.001). Using flow cytometry, we further determined that the lymph node cells, when cultured overnight with the specific tolerogen, evidenced an increase in surface expression of Ebi3 and surface expression TGFβ/LAP⁺ cells, predominantly among non-Treg cells. Specifically, we observed increased intracellular Ebi3 (“iEbi3”) (p<0.05) paralleled by a slight but significant increase in Foxp3⁺CD25⁺ Treg cells (p<0.01) in cultures incubated in the presence of soluble CBA antigens, but not when exposed to B6 or DBA/2 (third party) soluble antigens. However, sEbi3⁺ and surface TGFβ⁺ CD4+ T cells were detected in much greater numbers in 24 hour culture with CBA alloantigens on day 35 (p<0.001 and p<0.001, respectively). Using imaging flow cytometry (i.e., ImageStream® microscopy analysis system), sEbi3 appeared to be secreted as exosomes by the Treg cells and captured by bystander CD4 non-Treg memory T cells. The term bystander as used herein should be understood to mean cells that do not produce exosomes but rather passively acquire exosomes from exosome producers.

Spleen and lymph node cells freshly harvested on day 35 from the Ebi3-knockout Ebi3^{loxp}×Foxp3^{Cre} F1 mice showed a 10-fold lower level of sEbi3⁺ population than the level in the normal dual reporter mice. Ebi3 deletion in Foxp3 Treg cells eliminated the sEbi3 response, indicating that internal Ebi3 (iEbi3)-producing, Foxp3 Tregs are the primary source of IL-35 in allo-specific regulation. Ebi3 deletion in Foxp3 Treg cells also eliminated sEbi3 expression otherwise detectable following 24 hour in vitro culture of T cells in the presence of CBA antigens.

These results suggest that “non-producing” bystander memory CD4 T cells acquire Ebi3 from exosomes released from allo-specific Tregs developed during the donor-specific transfusion (DST) plus co-stimulatory blockade treatment and it is these non-producing memory CD4 cells that may amplify the regulatory response.

### Example 2: Directly Quantifying Treg Cells Specific for Self-Antigens or Donor Alloantigens

Materials and Methods:

Single cell suspensions of spleen, lymph node cells or peripheral blood mononuclear cells were cultured overnight in DMEM+10% FBS in the presence of a specific test antigen or crude cell lysate. When measuring alloantigen-specific induction of tolerance, the antigen preparation was a supernatant obtained from a sonicate of 10×10⁶ donor spleen, lymph node cells or peripheral blood mononuclear cells, centrifuged at 10,000×g for 14 minutes at 4° C. with phenylmethylsulfonyl fluoride (PMSF) and protease inhibitor iodoacetamide (IAA) at a concentration of 4×10⁷ cell equivalents per mL. To measure allospecific Treg, no intact donor cells are used. Both nanovesicles and soluble protein are present in these crude antigen preps. See e.g., Bracamonte-Baran and Burlingham, Biomed. J. 2015, 38(1):39-51.

When detecting self-antigen-specific Treg cells, the antigen preparations can contain purified proteins obtained from commercial sources. After an overnight culture of 1×10⁶ cells (e.g., PBMC or lymph node cells) and 10 μg/mL antigen (final concentration in DMEM+10% fetal bovine serum) per assay well, mononuclear cells were used for standard flow cytometry, CD3/CD4- or CD8-gated-readout. Alternatively, CD4+ or CD8+ T cells were used for a miniaturized assay with automated imaging.

Results

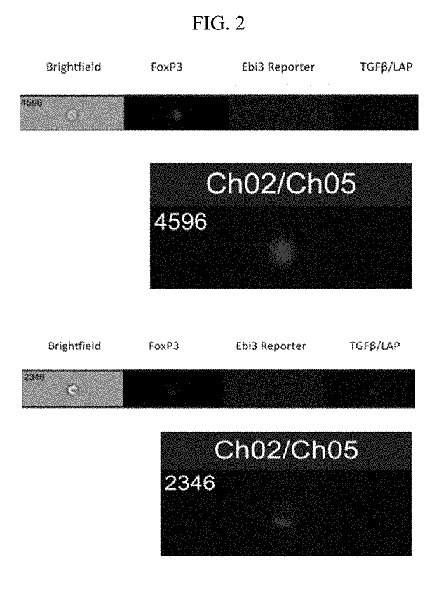

FIGS. 1 and 2 show readouts of a T-CBC assay using imaging flow cytometry. As shown in FIG. 1, Ebi3 was detected on the surface of Ebi3-transcriptionally active cells (left), as well as on Ebi3-transcriptionally inactive bystander cells (right). FIG. 1 suggests that in Ebi3-transcriptionally active Foxp3⁺ T cells, Ebi3 protein expression is uniform over the entire cell surface. The surface membranes of cells that do not themselves express Ebi3 (“non-Ebi3-producer cells”) were studded with round Ebi3⁺ exosome-like structures. Besides having distinctive imaging profiles, Ebi3-producer T cells were distinguishable using 2D flow cytometry from T cells that passively acquired surface Ebi3 in the dual reporter mice (see Example 1) (FIG. 1, left). To explore the basis of exosome acquisition in the T-CBC assay, in separate experiments (not shown), lymph node cells from tolerized B6 mice (in an upper well) were separated from naïve B6 Thy 1.1⁺ T cells (in a lower well) by a 1.0 μM pore-size semi-permeable membrane. After lymph node cells of the tolerized B6 mice were activated in the presence of a specific tolerogen, the naïve B6 Thy 1.1⁺ T cells acquired cell-bound Ebi3-containing exosomes after 24 hours. The acquisition of Ebi3-containing exosomes is shown at the right side of FIG. 1. We observed that both Foxp3⁺ T cells and non-Foxp3⁺ memory T cells can acquire exosomes.

The functional significance of cell-bound Ebi3 exosomes is yet to be determined. However, in further experiments (not shown), active Ebi3 expression and passive Ebi3 exosome acquisition, both normally seen after 24 hour culture of CBA-stimulated lymph node cells, were abolished in Foxp3⁺ Treg cells from CBA-tolerized Ebi3-knockout Ebi3^{loxp}×Foxp3^{Cre} F1 mice. This suggests either (1) that antigen-induced Foxp3⁺ Treg cells produced all the surface Ebi3, or (2) that expression of Ebi3 protein by Foxp3⁺ Treg cells was required for Ebi3 production by other non-Foxp3⁺ cells. Foxp3⁺ Treg cells make IL12α+ Ebi3 (component subunits of IL-35), but not p28+Ebi3 (component subunits of IL-27). However, we cannot exclude other possible binding partners for the surface Ebi3.

FIG. 2 shows the induced surface expression pattern of TGFβ/LAP in the same overnight culture system. The left panel (FIG. 2) shows a Foxp3⁺ TGFβ/LAP⁺ T_{reg} cell. The more common sTGFβ/LAP⁺ T cell variety (a/k/a Tₕ3) is Foxp3^{neg} (FIG. 2, right). Whether these populations are completely independent of, or interdependent with, each other remains to be determined.

In a T-CBC assay for detecting Treg cell activity, surface EBi3⁺ has a clear advantage over the surface TGFβ/LAP⁺ in terms of ability to amplify the antigen-specific T regulatory response signal, but expression of surface TGFβ/LAP⁺ may reflect actual numbers of antigen-specific Treg cells in the sample more accurately.

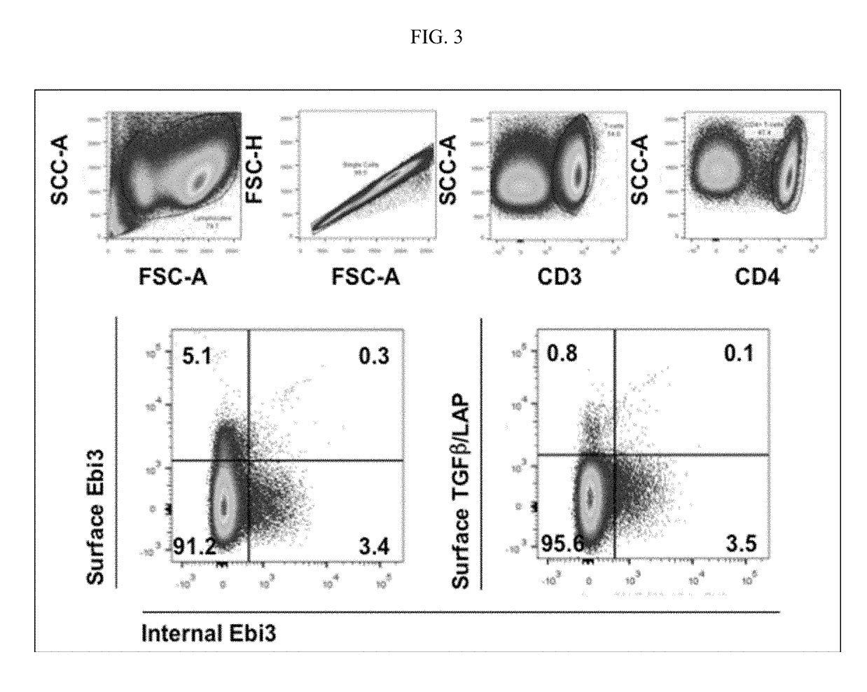

FIG. 3 quantifies the T-CBC results of FIGS. 1 and 2 using flow cytometry. These data suggest that surface Ebi3⁺ T cells belong to different CD4+ T cell subpopulations. These data also support the image analysis of FIG. 1, which demonstrates that the majority of sEbi3⁺ T cells displayed exosomes passively acquired from Ebi3-producer T cells. Even less correlation was observed between Ebi3 (intracellular) levels and sTGFβ/LAP. Notably, when corrected for media background, the latter responses in overnight cultures with specific tolerogen were both in the low (<1%) range. This range likely reflects the true frequency of Ag-specific Treg.

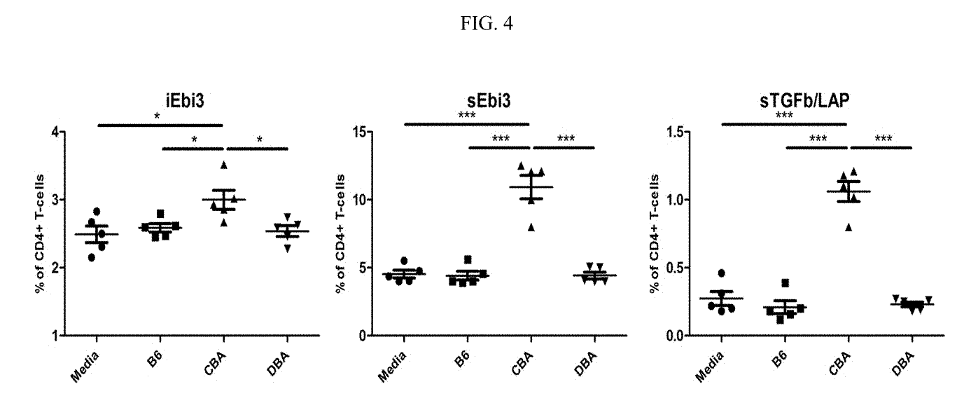

T cell-based assay for cell-bound cytokines (T-CBC): Each data point in FIG. 4 represents a single mouse. FIG. 4 shows that overnight culture of lymph node cells harvested from YFP-Foxp3/TdTomatoRed-Ebi3 dual reporter B6 mice 35 days after DST+ anti-CD40L tolerization with donor CBA crude antigen prep resulted in an intracellular Ebi3 synthesis increase (left) and a 10-fold higher increased expression sEbi3 (middle). In particular, we observed net increases of 8.0% vs. 0.8% over media control or self-antigen (Ag) cultures. There was also increased surface expression of TGFβ/LAP amongst lymph node CD4 T cells in response to donor CBA antigen. No increases in T-CBC were observed in cultures stimulated with self or 3rd party antigen preps.

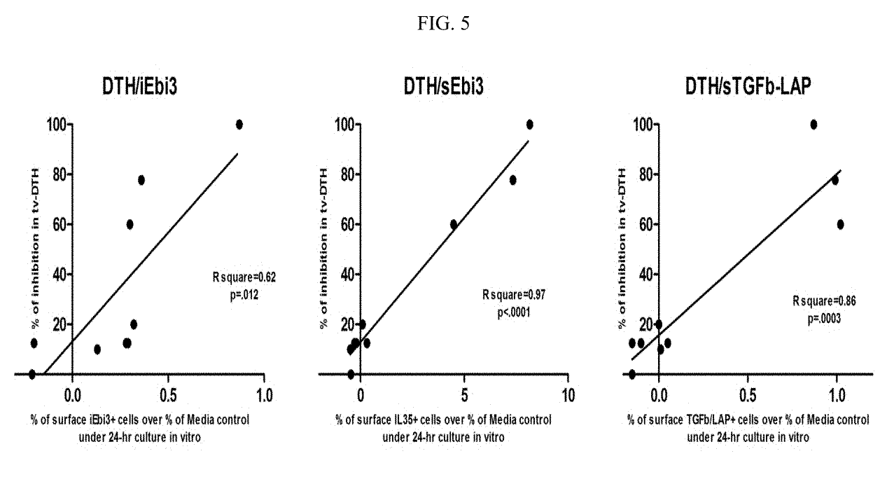

The results of every T-CBC assay test of mouse lymph node cells were compared with responses of spleen cells to donor, self, and 3rd party antigens in the tv-DTH linked suppression assay. Referring to FIG. 5, we also compared percentages of linked suppression as determined by a tv-DTH swelling response with percentages of linked suppression as determined by intracellular Ebi3 and T-CBC responses. T-CBC and intracellular Ebi3 are expressed on the y-axis as the percentage of CD4+ T cells, after media background is subtracted. Interestingly, the percentage of cells with an intracellular signal for Ebi3 transcription yielded a non-linear, S-shaped correlation, while the percentage of CD4+ T cells positive for surface Ebi3 gave a tight linear correlation with tv-DTH/% LS. The surface TGFβ/LAP⁺ T-CBC values correlated slightly less well with tv-DTH suppression, but over a much narrower (0-1% vs. 0-10%) dynamic range. The significance of these results is that the T-CBC assay, using surface Ebi3/IL-35 expression as the readout, provides the best correlate with the tv-DTH linked suppression assay.

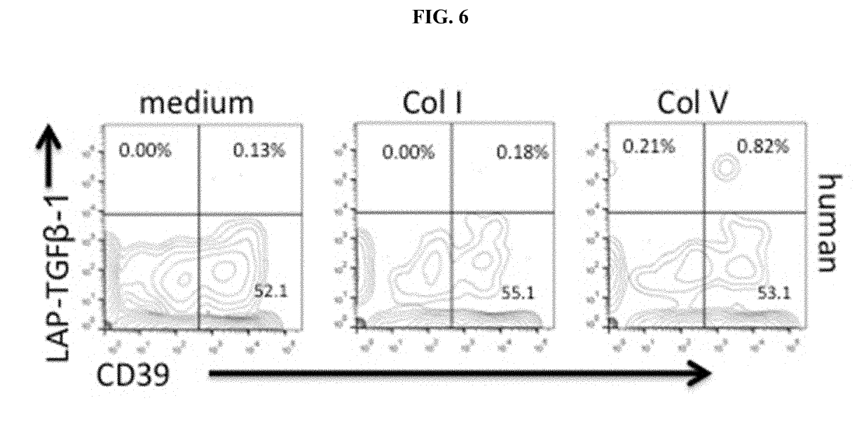

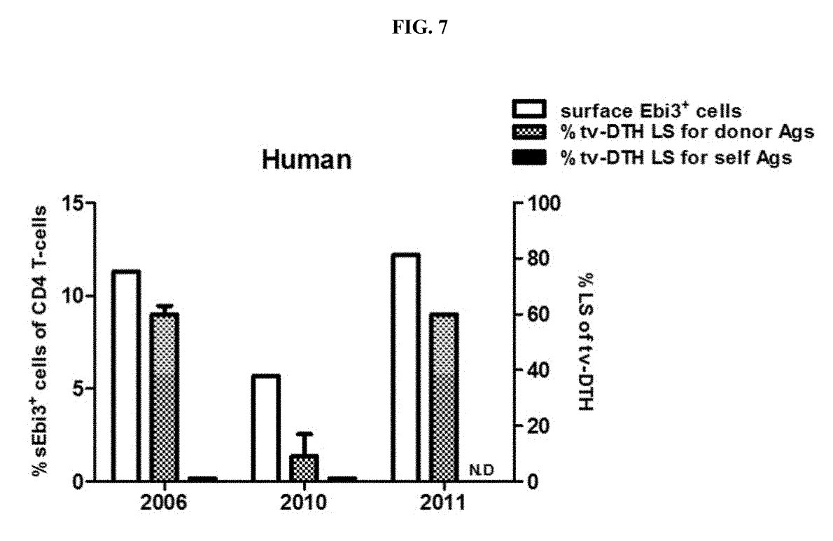

FIGS. 6 and 7 provide preliminary human Treg data in support of the relevance of the mouse results demonstrated in FIGS. 1-5 to human transplant recipients. FIG. 6 presents human Treg data for a lung transplant patient. This patient had developed strong regulation to a self-antigen, collagen type V, as determined by tv-DTH analysis. We observed that this regulation was reversible by a neutralizing antibody to TGFβ. Shown in FIG. 6 is a 2-dimensional flow cytometry diagram of the transplant patient's PBMC after overnight in vitro culture. The y axis presents T-CBC data collected using fluorescently labeled LAP-TGFβ, while the x-axis measures the fluorescence from detection of CD39, an extracellular ATPase known to be involved in regulation of the T cells that respond to human collagen type V. As shown, the culture revealed a small but distinct population of 0.82% of T cells positive for both markers (e.g., LAP-TGFB+/CD39+), but only in the culture stimulated with Collagen V. Culture in the presence of human Collagen type I did not result in LAP-TGFB+/CD39+ T cells. These data demonstrated a Collagen V-specific regulatory T cell response in this lung transplant subject.

FIG. 7 presents data for a T-CBC assay performed using PBMC collected from a kidney transplant patient exhibiting “metastable tolerance,” which means the patient exhibits alternating periods of strong regulation and poor regulation in the absence of any immunosuppressive drug therapy. The patient's peripheral blood was sampled at 3 different time points and analyzed by T-CBC assay for expression of surface Ebi3⁺ cells as a percentage of total CD4+ T cells, as well as by tv-DTH assay. As shown in FIG. 7, the T-CBC values for surface Ebi3⁺ cells as a percentage of total CD4+ T cells fluctuated, with >10% values at year 2006 and 2011, and a low value (˜5-6%) at the 2010 time point. The time points of highest T-CBC values correlated with strong (60%) values of linked suppression in the footpad tv-DTH inhibition assay. In summary, these data support a conclusion that the T-CBC assay is an excellent proxy for the tv-DTH linked suppression assay for studying tolerance and immune suppression in human subjects as well as animals.

### Example 3: IL-35 Surface Staining

Single cell suspensions were prepared from spleen, lymph nodes, or other tissues of interest. Staining for live and dead cells was performed prior to surface staining with IL-35 antibody. LIVE/DEAD® fixable dead cell stain Aqua (Invitrogen) was used at a dilution of 1:700 in phosphate buffered saline. Alternatively, scatter gates can be used to exclude apoptotic cells and doublets. After washing off the live/dead stain, we prepared a staining cocktail for surface markers for staining different lymphocyte populations based on the experimental needs. A Foxp3 reporter mouse was used for Treg analysis. A staining cocktail composition for detecting surface expression of IL-35 on Treg/Teff cells is listed below:

NMS (10%) of staining buffer (FACS) volume

CD4 (at 1:500 dilution)

CD25 (at 1:500 dilution)

CD45RB (at 1:500 dilution)

Ebi3 Biotin-linked antibody (V1.4F5.29 clone described by Collison et al, Nature Immunol., 2010). The stock concentration of V1.4F5.29 antibody was 2 mg/ml. V1.4F5.29 antibody was used at a 1:50 to 1:100 dilution.

Cells were incubated with antibodies at 4° C. for 15-20 minutes and then centrifuged. Excess antibody was washed from the stained cells using FACS buffer (1-2×). Secondary staining was performed for Ebi3 using Streptavidin-APC/Streptavidin-PacBlue, which have low background relative to other conjugated secondary antibodies (e.g., R-phycoerythrin (PE)/Cy, PE-Cy™7, APC-Cy™7, Brilliant™ Ultraviolet (BUV)) and can be used at a dilution of 1:200 to 1:500.

Secondary antibody was incubated with the primary Ab stained cells for 15-20 minutes at 4° C., and then centrifuged. Excess secondary antibody was washed off using FACS buffer (1-2×). The resulting samples were ready for flow analysis and, alternatively, for fixation and permeabilization for other applications (e.g., staining for transcription factors or other cytokines).

### REFERENCES

1. VanBuskirk, A. M., et al., Human allograft acceptance is associated with immune regulation. J Clin Invest, 2000. 106(1): p. 145-155.
2. Levings, M. K., et al., Human CD25+CD4+ T suppressor cell clones produce transforming growth factor beta, but not interleukin 10, and are distinct from type 1 T regulatory cells. J Exp Med, 2002 Nov. 18. 196(10): p. 1335-46.
3. Mascanfroni, I. D., et al., Metabolic control of type 1 regulatory T cell differentiation by AHR and HIF1-alpha. Nat Med, 2015.
4. Olson, B. M., et al., Human prostate tumor antigen-specific CD8+ regulatory T cells are inhibited by CTLA-4 or IL-35 blockade. J Immunol, 2012. 189(12): p. 5590-601.
5. Olson, B. M., J. A. Sullivan, and W. J. Burlingham, Interleukin 35: a key mediator of suppression and the propagation of infectious tolerance. Front Immunol, 2013. 4: p. 315.
6. Torrealba, J. R., et al., Metastable tolerance to rhesus monkey renal transplants is correlated with allograft TGF-beta 1+CD4+ T regulatory cell infiltrates. J Immunol, 2004. 172(9): p. 5753-64.
Xu, Q., et al., Human CD4+CD25 low adaptive T regulatory cells suppress delayed-type hypersensitivity during transplant tolerance. J Immunol, 2007. 178(6): p. 3983-95.
8. Howard, A. L., Pezzi, H. M., Beebe, D. J., Berry, S. M. Exclusion-Based Capture and Enumeration of CD4+ T Cells from Whole Blood for Low-Resource Settings. J. Lab. Autom., 2014. 19(3): p. 313-321.
9. Berry, S. M., Singh, C., Lang, J. D., Strotman, L. N., Alarid, E. T., Beebe, D. J. Streamlining gene expression analysis: integration of co-culture and mRNA purification. Integr. Biol. 2014. 6(2): p. 224-231.
10. Casavant, B. P., Guckenberger, D. J., Beebe, D. J., Berry, S. M. Efficient Sample Preparation from Complex Biological Samples Using a Sliding Lid for Immobilized Droplet Extractions. Anal. Chem. 2014. 86(13): p. 6355-6362.

The present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

We claim:

1. An in vitro method of detecting antigen-specific immune suppression in a subject, the method comprising:
(a) culturing T cells of the subject for about 24 hours in the presence of one or more target antigens; and
(b) detecting in the cultured T cells expression of a marker that indicates antigen-specific regulatory T cell response in the subject, wherein detecting expression of the marker in the population of cultured T cells indicates antigen-specific immune suppression in the subject.

2. The method of claim 1, wherein the T cells are obtained from a biological sample selected from the group consisting of lymph nodes, peripheral blood, and splenocytes.

3. The method of claim 1, wherein the marker is selected from the group consisting of Ebi3 and TGFβ/LAP.

4. The method of claim 1, wherein step (b) is carried out by measuring the proportion of cells positive for surface expression of Ebi3 among CD4-positive or CD8-positive T cells of the cultured T cells.

5. The method of claim 1, wherein step (b) is carried out by measuring the proportion of cells positive for intracellular expression of TGFβ/LAP among CD4-positive or CD8-positive T cells of the cultured T cells.

6. The method of claim 1, wherein the one or more target antigens comprise self-antigens and the antigen-specific linked immune suppression is associated with an autoimmune disease.

7. The method of claim 1, wherein the one or more target antigens comprise alloantigens and the antigen-specific linked immune suppression is associated with a cell, tissue, or organ transplant.
